(19) United States
(12) Patent Application Publication
(10) Pub. No.: US 2022/0362537 A1
(43) Pub. Date: Nov. 17, 2022

(54) Flow modulation devices and methods of use with a hemodyalisys fistula or a graft

(71) Applicant: Farajallah Mounir Hanna al-kass, Glendale, CA (US)

(72) Inventor: Farajallah Mounir Hanna al-kass, Glendale, CA (US)

(21) Appl. No.: 17/750,179

(22) Filed: May 20, 2022

Related U.S. Application Data
(60) Provisional application No. 63/188,436, filed on May 13, 2021.

(51) Int. Cl. A61M 39/02; A61M 1/36; A61M 1/14

(52) U.S. Cl. CPC A61M 39/0208; A61M 1/3655; A61M 1/3656; A61M 1/14; A61M 2039/0226

(57) ABSTRACT

A flow modulation device is positioned around a hemodialysis fistula or a graft and includes a non-expandable outer jacket with an inwardly-inflatable flow-modulation chamber positioned inside thereof. The flow modulation chamber is configured to be in fluid communication with a control chamber via a flexible catheter. The control chamber includes a puncture-resistant housing having an inner cavity covered by a self-sealing elastic membrane sealingly attached thereto and configured for repetitive needle punctures. Upon injection of fluid into the control chamber via a needle puncture, the flow modulation chamber inflates inwardly to compress the hemodialysis fistula or the graft causing a reduction of blood flow therethrough.

### BACKGROUND

Without limiting the scope of the invention, its background is described in connection with implantable devices used for facilitating kidney hemodialysis. More particularly, the invention describes an implantable flow modulation device positioned around an arteriovenous fistula or graft and methods of using the device for modulating blood flow therethrough.

The main kidney function is to remove the byproduct wastes and the excess fluids from the blood and excrete them via the urine. Unfortunately, many patients suffer from a chronic kidney disease leading to a significant impairment of kidney function. Patients with diabetes, high blood pressure, or a family history of kidney disease are more prone to developing chronic kidney disease. A small percentage but a large number of chronic kidney disease patients in stage IV will eventually progress to complete kidney failure. This means that the kidneys do not remove enough wastes and excess fluid from the blood flow. At this point, the dialysis treatments are prescribed to take over the function of the kidney. Dialysis is not a cure for kidney disease. A kidney transplant is the only alternative method for treating patients with kidney failure.

There are two known types of dialysis: hemodialysis and peritoneal dialysis.

Hemodialysis is the most common method for dialysis. The present invention has useful applications in the hemodialysis setting. There are approximately 500,000 patients with an advanced form of chronic kidney disease in the United States that receive hemodialysis treatments.

A hemodialysis machine (also called an artificial kidney) removes wastes, and excess fluids from the bloodstream in the body by circulating the patient's blood through the machine. Hemodialysis patient needs to have hemodialysis access or vascular access (also called arteriovenous access) created by a surgical procedure to allow a large portion of the blood flow to be diverted during the time of the procedure to circulate through the dialysis machine.

There are two common types of arteriovenous accesses: a fistula and a graft. A dialysis fistula is the first common method to create vascular access and increase blood flow volume locally by a direct, surgically created connection between an artery and a vein. A dialysis graft is the second method to create vascular connection by using a synthetic tubular fabric (such as made from PTFE or Dacron) to join an artery to a vein. The forearm or the upper arm is usually used to create the dialysis fistula or graft, such as using the brachial or the radial arteries and adjacent veins. For the purposes of this description, the term “arteriovenous connection” is used to describe either the arteriovenous fistula or the graft placed to connect the artery to the vein for the purposes of facilitating hemodialysis.

Blood flows from the artery, through the fistula or the graft, and into the vein. To connect the patient to a dialysis machine, two large hypodermic needles are inserted through the skin and into the fistula. Blood is removed from the patient through one needle, circulated through the dialysis machine, and returned to the patient through the second needle. Typically, patients undergo hemodialysis approximately four hours a day, three days a week.

Various problems, however, have been experienced with the use of an arteriovenous fistula or graft. For example, arterial “steal” occurs when excessive blood flow through the arteriovenous graft “steals” blood from the distal arterial bed. The steal syndrome is a complication that might happen secondary to the creation of an arteriovenous connection. The incidence of the steal syndrome is reported as high as 8% in the current vascular literature. The steal syndrome occurs secondary to the retrograde flow of blood from the distal aspect of the artery (located distal to the arteriovenous fistula or graft) into the vein through the fistula. The steal simply leads to a shortage in the arterial supply (oxygen and nutrients) to the organs (usually the arm) located distal to the arteriovenous connection. Hence, symptoms of steal syndrome progress from painless cool extremity to claudication (cramping), rest pain, and finally to tissue necrosis (tissue death and gangrene).

Ischemic neuropathy is most common in diabetic patients. The frequency of developing ischemic neuropathy reaches up to 10% after the creation of an arteriovenous connection. The symptoms present as severe pain, sensory loss, motor weakness, and eventually paralysis of the muscles innervated by the nerves distal to arteriovenous connection.

In addition to the arterial steal phenomenon, a greater cardiac output is required to compensate for the shunted blood flow through the fistula or graft. In some patients, their native heart cannot continuously provide this high output. A large volume of blood flows from the artery 100 into the vein 300 through the arteriovenous connection 180 back to the heart as illustrated in FIG. 1 and FIG. 3. To compensate for additional returned blood volume (volume overload) thru the bypass detour/connection, the heart pumps more forcefully and more rapidly, thus greatly increasing its output of blood. Eventually, the increased effort may strain the heart and leads to heart failure. The high-output heart failure may therefore develop secondary to kidney failure, but this occurs not as frequently as the steal syndrome.

When addressing the problems of high-output heart failure, ischemic neuropathy, and dialysis-associated steal syndrome, the clinician is faced with the dilemma of preventing the progression of the sequelae of these problems but not at the expense of losing the vascular access. So, the solutions should be directed towards decreasing the blood flow into the venous side. However, a decrease in blood flow in the vein may limit the capacity of the fistula to sustain the high throughput required for hemodialysis as well as increase the risk of fistular or graft thrombosis.

Various other complications can also occur. For instance, the blood flowing through the arteriovenous fistula or graft can often reach turbulent flow rates. This stream of fast-moving blood then exits the arteriovenous graft and contacts the vein connected to the graft. This collision between the flow of blood and the vein may cause the development of neointimal hyperplasia which leads to the thickening of the vein walls and a narrowing of the vessel. As the vein narrows, flow through the arteriovenous fistula or graft decreases and blood within this passage may ultimately clot.

Many review articles highlight a plethora of complications associated with the creation of an arteriovenous connection. One example is that by Stolic R. Most Important Chronic Complications of Arteriovenous Fistulas for Hemodialysis, Med Princ Pract 2013; 22:220-228, as found at https://doi.org/10.1159/000343669.

Present solutions for reducing excessive blood flow through the arteriovenous connection are divided into two categories: surgical and endovascular interventions. The surgical intervention consists of banding of the arteriovenous connection (Miller Banding procedure) or revision of the arteriovenous connection (DRIL and RUDI procedure). The Miller banding procedure uses sutures to create a band around part of the venous outflow of the vascular access. The band increases the resistance on the venous side, adjusts blood flow through the arteriovenous connection, and hence improves circulation to the distal arm. The distal revascularization and interval ligation (DRIL) and the revision using distal inflow (RUDI) procedures both allow treatment of the steal with various degrees of success. Both procedures are complex surgical interventions that carry their own risks and complications.

The endovascular intervention is performed percutaneously via a small skin incision under X-ray guidance. Two common procedures are described in the literature. The first procedure is to deploy a large stent graft within a smaller bare stent in the venous outflow. The second procedure is to deploy a stent graft with a constricting segment (created by balloon inflation) in the venous outflow. Both techniques make the diameter of the venous outflow smaller.

Both the surgical and endovascular approaches achieve some success but are still far from satisfactory because of the following reasons:
a. All these solutions modulate the flow of blood into the venous system in an approximate way. The surgical intervention revises the revascularization of the dialysis fistula and results in a subjective reduction of the blood flow into the vein. Similarly, the endovascular intervention subjectively reduces the flow into the vein by creating a constricting segment in the deployed stent. Both interventions use a rough estimate for adjusting the flow into the vein.
b. The dialysis apparatus (artery, vein, arteriovenous connection) is always changing. In that sense, performing a single corrective intervention does not offer a lasting solution. If the extent of blood flow into the vein is satisfactory at one point, it might not be the case in a few weeks or months. Dialysis patients are at higher risk of developing atherosclerosis and narrowing their arteries. If the feeding artery to the dialysis fistula or graft gets stenotic, the blood flow in this artery is reduced, which may render the fistula not suitable for dialysis. Similar scenarios might occur in weeks and months on the venous side. Aneurysmal dilation and stenosis frequently occur in the venous outflow of the fistula, and these changes lead to the redistribution of the blood flow through the arteriovenous connection. To compensate for the new situation, patients may need another complex corrective surgery.
c. Although these surgical and endovascular interventions are considered minor surgeries and are performed under local anesthesia (some need conscious sedation), they still have their own risks and complications. These risks may be exacerbated since most dialysis patients have other associated medical conditions and co-morbidities that contribute to the risk profile of these interventions.

Presently, there is no lasting solution to readjust the flow in the dialysis fistula or graft because the current solutions do not consider the changing nature of the blood flow through the fistula over time (such as evolving stenosis in the arteries and veins as well as aneurysmal dilation in the veins) that contribute to the redistribution of the blood flow after a single corrective intervention.

In view of the above drawbacks, there is a need for arteriovenous grafts and methods that can prevent and minimize these complications over the entire course of hemodialysis, which may take many years.

### SUMMARY

Accordingly, it is an object of the present invention to overcome these and other drawbacks of the prior art by providing a novel flow mediation device and a method for its use in regulating shunt blood flow through an arteriovenous connection created to facilitate hemodialysis treatments.

It is another object of the present invention to provide a novel device and method for reducing shunt blood flow in between dialysis treatments.

It is a further object of the present invention to provide a novel device and a method of modulating shunt blood flow through the arteriovenous connection to reduce a risk of a steel syndrome and high-output heart failure.

A flow modulation device of the present invention is positioned around a hemodialysis fistula or a graft. The flow modulation device may include a non-expandable outer jacket with an inwardly-inflatable flow-modulation chamber positioned inside thereof. The flow modulation chamber may be configured to be in fluid communication with a control chamber via a flexible catheter. The control chamber may include a puncture-resistant housing having an inner cavity covered by a self-sealing elastic membrane sealingly attached thereto and configured for repetitive needle punctures. Upon injection of fluid into the control chamber via a needle puncture, the flow modulation chamber inflates inwardly to compress the hemodialysis fistula or the graft causing a reduction of blood flow therethrough.

A novel method of modulating shunt blood flow through an arteriovenous connection facilitating hemodialysis may include the following steps:
a. subcutaneously positioning a flow modulation device comprising a non-expandable outer jacket with an inwardly-inflatable flow modulation chamber positioned inside thereof around the arteriovenous connection,
b. subcutaneously positioning a control chamber fluidly connected to the flow modulation chamber via a flexible catheter, the control chamber comprising puncture-resistant housing having an inner cavity covered by a self-sealing elastic membrane sealingly attached thereto and configured for repetitive needle punctures,
c. gaining access to the control chamber via a needle puncture, and
d. injecting or withdrawing fluid into or out of the control chamber, thereby inflating or deflating the flow modulation chamber around the arteriovenous connection,
   whereby inflation of the flow modulation chamber causing the flow modulation device to compress the arteriovenous connection and reduce blood flow therethrough.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings, in which:

FIG. 1 is a diagram view of the present invention showing a flow modulation device positioned around an arteriovenous connection.

FIG. 2 is the same as in FIG. 1 but with the flow modulation device inflated to reduce blood flow through the arteriovenous connection.

FIG. 3 is a close-up general view of the flow modulation device positioned around the arteriovenous connection.

FIG. 4 is the same as in FIG. 3 but shows the flow modulation device in its inflated state.

FIG. 5 is a general view of the implantation and subcutaneous positioning of the flow modulation device and the control chamber attached thereto in the arm of a patient.

FIG. 6 is a close-up of the device and its control chamber seen in FIG. 5.

FIG. 7 is the same as in FIG. 5 but with a portion of the skin removed for clarity of the illustration.

FIG. 8 is a general view of the flow modulation device in its unwrapped state connected to the control chamber.

### DETAILED DESCRIPTION OF PREFERRED EMBODIMENTS OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and make part of this disclosure.

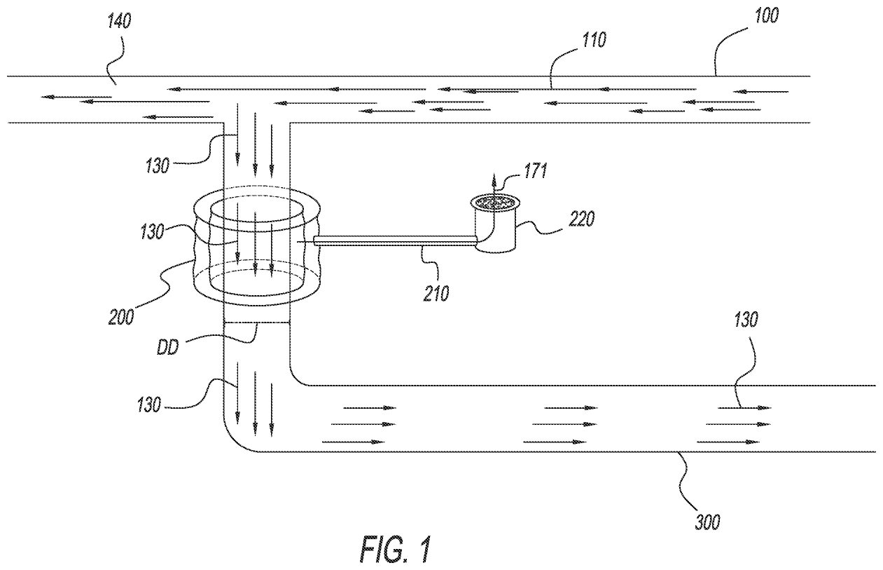

FIG. 1 shows a general diagram of the blood flow in the vascular system of the patient with the addition of an arteriovenous connection 180 for the purposes of facilitating hemodialysis. Normal arterial flow 110 through an artery 100 is divided into a blood flow portion 140 perfusing the distal portion of the arm of the patient, and the shunt flow 130 through the arteriovenous connection and proceeding directly to the outflow vein 300. As mentioned above, when the shunt flow 130 reaches excessive levels, the patient may suffer from “steal” consequences including the complications of under-perfusion of the arm and even heart failure resulting from attempts to overcompensate for the shunted blood flow 130.

The main concept behind the current invention is to modulate the shunt blood flow 130 into the dialysis fistula or a graft based on demand, namely to increase the shunt flow 130 just before and during the dialysis and decrease it afterward and in between dialysis sessions. Assuming that the patient undergoes dialysis for 3 hours three times per week, the total duration of time when the shunt flow needs to be high to facilitate proper operation of the dialysis machine is 4×3=12 hours out of a total of 24×7=168 hours in a week, or only during less than 7% of the time (12 hours out of 168 hours). The flow modulation device 200 is devised to provide for such flow adjustment before and after one, several, or all dialysis procedures by reducing the maximum allowable opening DD corresponding to the diameter of the arteriovenous connection or releasing thereof as required. In other circumstances, the flow modulation device of the invention may also be used less frequently to simply increase or decrease continuous shunt flow 130 through the arteriovenous connection, such as on a weekly, semi-monthly, monthly, or some other long-term basis, or on an as-needed basis as determined by the treating physician.

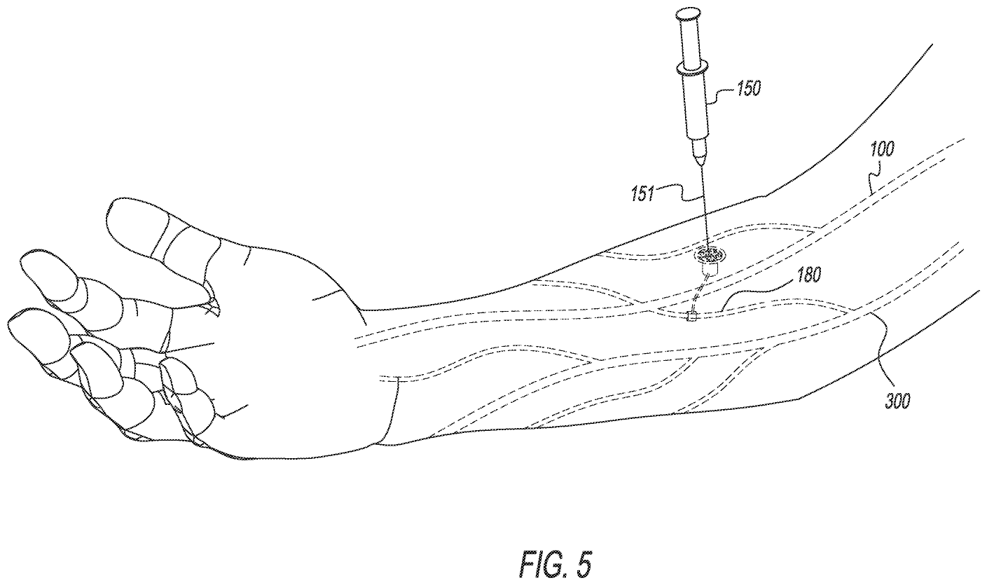

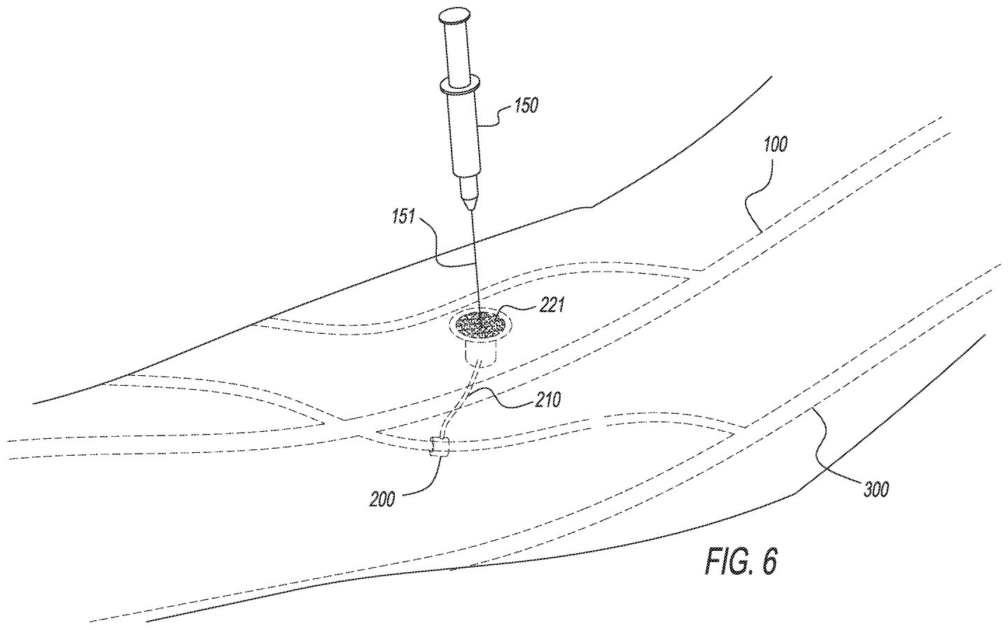

Various views of the flow modulation device 200 and different states of operation thereof are seen in FIGS. 1-8 and described in greater detail below.

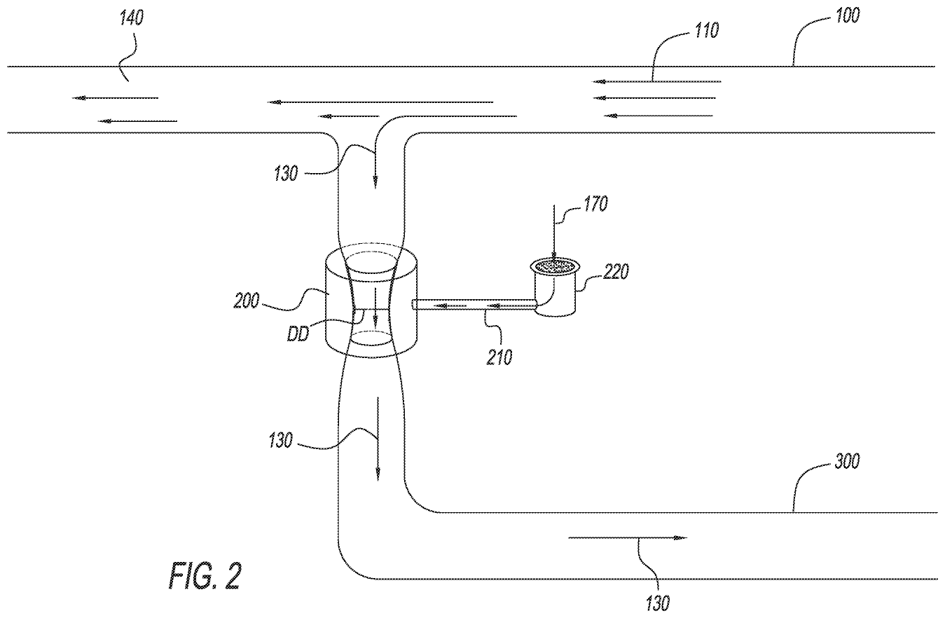

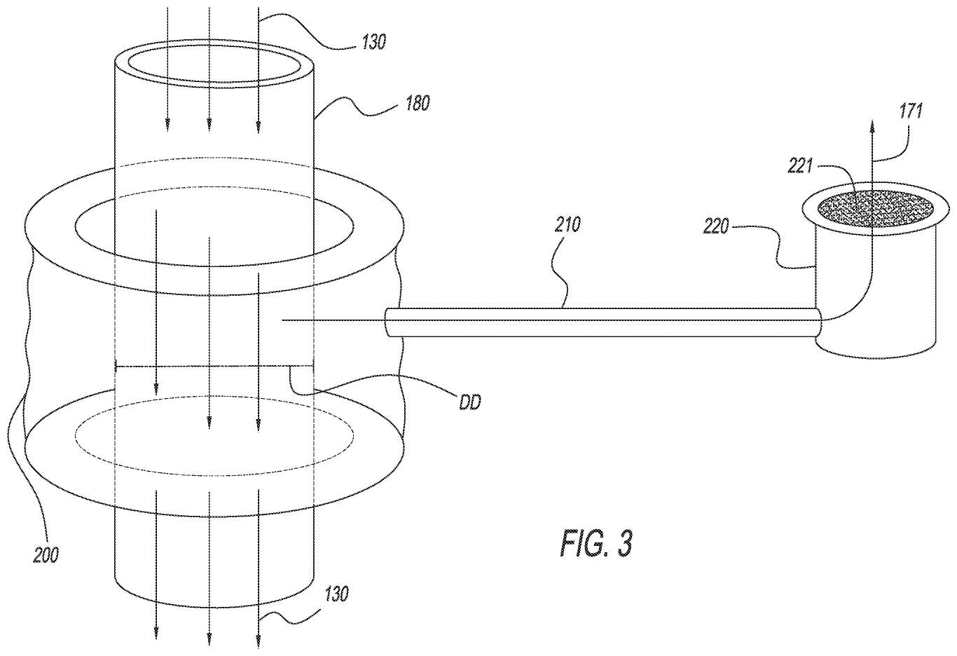

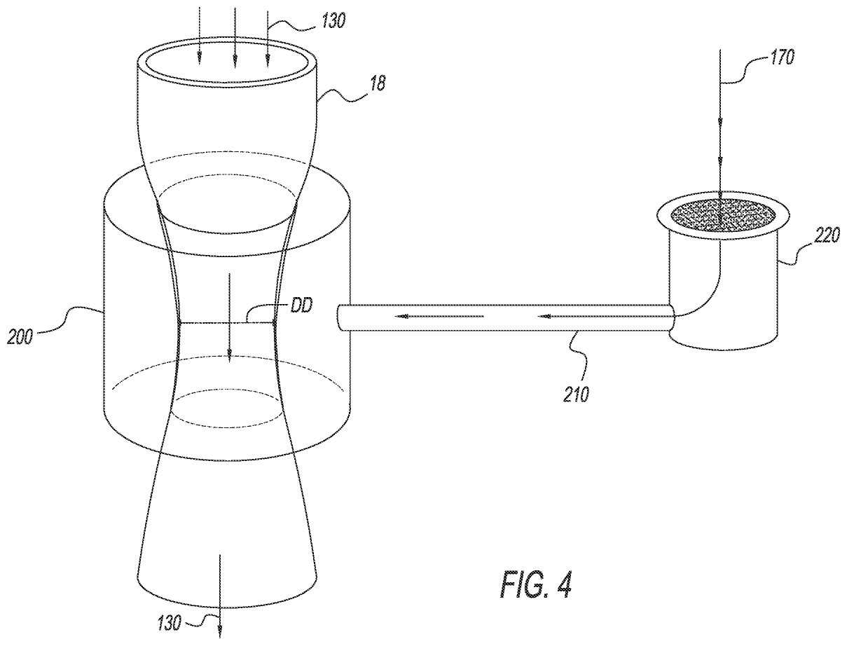

In broad terms, the flow modulation device includes an inwardly-inflatable flow modulation chamber fluidly connected via the catheter 210 to the control chamber 220. The control chamber 220 in turn includes a self-sealing membrane 221 configured to allow injection or removal of fluid therethrough via a needle 151 at the end of a syringe 150. Injection of fluid 170 through the membrane 221 causes inward inflation of the flow modulation chamber of the device 200 and narrowing of the diameter DD (seen in FIGS. 2 and 4), causing a reduction in the shunt blood flow 130. Withdrawal of fluid through the membrane 221 causes deflation of the flow modulation chamber and expansion of the diameter DD in the arteriovenous connection 180—leading to an increase in the shunt flow 130 (FIGS. 1 and 3).

The flow modulation device 200 comprises generally a non-expandable outer jacket positioned around the arteriovenous connection 180 and an inwardly-inflatable flow modulation chamber located circumferentially between the outer jacket and the connection 180. In its deflated state, the flow modulation chamber is sized to not occlude or obstruct the flow of blood through the arteriovenous connection 180 as seen in FIG. 1 and FIG. 3. Since the arteriovenous connection may come in different sizes, a number of sizes for the flow modulation device 200 may be offered, for example, ranging in internal diameter from about 2 mm to about 15 mm, such as at least 2 mm, at least 3 mm, at least 3 mm, at least 5 mm, at least 6 mm, at least 7 mm, at least 8 mm, at least 9 mm, at least 10 mm, at least 11 mm, at least 12 mm, at least 13 mm, at least 14 mm, or at least 15 mm. In other embodiments, just a few or even a single universal flow modulation device 200 may be offered that allows a broader range of inflation of the flow modulation chamber that would cover a range of diameters of the arteriovenous connection 8.

The outer jacket of the flow modulation device may be made from a suitable biocompatible plastic approved for long-term implantation, such as polyurethane, polypropylene, Teflon, or a similar appropriate material. The flow modulation chamber may be made from an implantable-grade biocompatible expandable polymer membrane, such as other forms of polyurethane, silicone, natural or synthetic rubber, etc. dimensions and thickness of the flow modulation chamber may be selected to provide a gentle narrowing of the fistula or graft connection 180 upon inflation as seen in FIG. 4 so as to avoid stagnation zones and reduce the risk of thrombus formation in the connection 180.

In some embodiments, the flow modulation device may be made in a generally circular, toroidal or hollow cylindrical shape as seen in FIGS. 3 and 4. It is intended to be placed around the fistula or graft 180 during the same surgery as used for the formation of the arteriovenous connection 180. In other embodiments, the flow modulation device 200 may be made with a C-shaped cross-sectional shape (not shown in the drawings) so as to allow adding the device to the existing arteriovenous connection by flattening the connection 180 and sliding it inside the flow modulation chamber after the course of hemodialysis treatments have already started. This use may be advantageous for patients that start to experience “steal” syndrome after being on dialysis for some time.

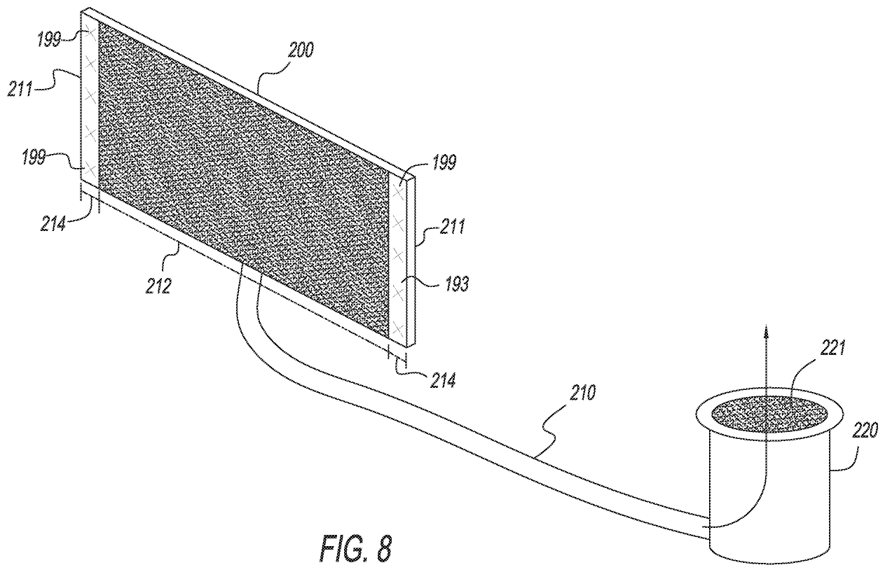

In further embodiments, an initially unfolded or generally flat device 200 is seen in FIG. 8 and comprises an outer jacket with an inflatable chamber 212 in the central portion thereof. Two strips 211 may be located on both opposing sides of the non-expandable outer jacket with the inwardly inflatable chamber 212 located between thereof to allow the device 200 to be wrapped around the arteriovenous connection 180. Once approximated or overlapped with each other, the strips 211 may be sutured or stapled together using sutures 199, or otherwise fused, snapped, or joined together. The width 214 of each strip 11 may range from about 1 mm to about 10 mm, such as at least 1 mm, at least 2 mm, at least 3 mm, at least 3 mm, at least 5 mm, at least 6 mm, at least 7 mm, at least 8 mm, at least 9 mm, or at least 10 mm. This embodiment may also be used during a secondary surgery, sometime after the initial fistula or graft is first inserted. One advantage of this embodiment is greater size flexibility to cover a broader range of arteriovenous connections with a single flow modulation device 200.

The control chamber 220 may be made from a rigid, puncture-resistant, implant-grade biocompatible polymer or metal. The housing of the control chamber may be made with an inner cavity covered by a self-sealing elastic membrane 221 sealingly attached thereto and configured for repetitive needle punctures. The design of a control chamber 220 may be similar to that of subcutaneously implantable fluid injection vascular access ports. One example of a suitable design is described in the U.S. Pat. No. 5,137,529 incorporated herein by reference in its entirety. One advantageous difference of the control chamber of the present invention is that the size and volume thereof may be smaller than the size of a conventional subcutaneous port as it does not need to support high flow rates of fluids therethrough.

The catheter 210 fluidly connecting the flow modulation device 200 and the control chamber 220 may also be made from a biocompatible implantable grade polymer such as polyurethane. The catheter 210 may be flexible yet resistant to kink or collapse, especially when a vacuum is applied to the control chamber 220. In some embodiments, the catheter 210 may be made with a wall reinforced by a wire or a fiber forming a braid or another suitable reinforcing arrangement. The size of the internal diameter for the catheter 210 may range from about 0.5 mm to about 5 mm, such as at least 0.5 mm, at least 1 mm, at least 2 mm, at least 3 mm, at least 4 mm, or about 5 mm. the wall thickness may range from about 0.25 mm to about 3 mm, such as at least 0.25 mm, at least 0.5 mm, at least 1 mm, at least 1.5 mm, at least 2 mm, at least 2.5 mm, or about 3 mm. The length of the catheter may be from about 10 mm to about 100 mm, such as at least 10 mm, at least 20 mm, at least 30 mm, at least 40 mm, at least 50 mm, at least 60 mm, at least 70 mm, at least 80 mm, at least 90 mm, or about 100 mm.

All three components of the flow modulation system, namely the flow modulation device 200, the catheter 210, and the control chamber 220 may be provided as a single unit or may be provided individually, in which case the exit port of the control chamber 220 and the inlet port of the flow modulation device 200 may be equipped with suitable barbed fittings configured for assembly of the system prior to implantation. One advantage of providing components of the system individually is that the same control chamber may be used with different size flow modulation devices 200.

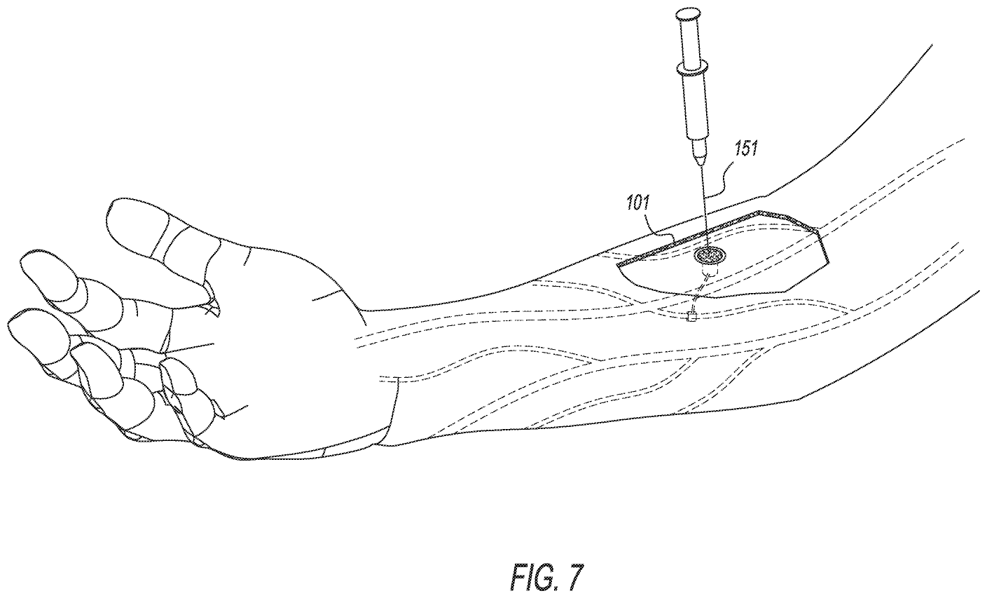

Implantation of the device may be done at the same time as the formation of the fistula or insertion of the arteriovenous graft as seen in FIG. 7. In other embodiments, implantation of the device 200 may be done in a separate surgery sometime after the formation of the initial arteriovenous connection 180. In any case, all components of the system are positioned under the skin layer 101. The flow modulation device 200 is positioned around a section of the arteriovenous connection 180 and the control chamber 220 may be positioned nearby and closely under the skin 101 so as to be easily identifiable by palpation or X-Ray. The connecting catheter 210 may be placed between the flow modulation device 200 and the control chamber as appropriate based on the patient's anatomy. Once all components of the system are connected together and placed appropriately in the patient's arm, a needle 151 and a syringe 150 may be used to remove all air from the internal volume of the system and replace it with saline or another suitable biocompatible fluid.

In use, a needle puncture may be used to gain access to the internal cavity of the control chamber 220. If a reduction of shunt blood flow 130 through the connection 180 is desired, fluid may be injected into the control chamber 220, which would cause gradual inward inflation of the flow modulation device 200 and a subsequent reduction in the size of the opening DD inside thereof. That creates additional resistance to the shunt blood flow 130 causing its gradual reduction. If the flow of blood needs to be increased, needle 151 may be used to withdraw the fluid from the control chamber 220 and subsequently cause a gradual deflation of the flow modulation device 200 and expansion of its internal diameter. This, in turn, causes a release of the restriction in the arteriovenous connection 180 leading to an increase in the internal diameter DD thereof and the desired increase in the shunt blood flow 130.

One advantage of the present invention is that the process of increasing or decreasing the shunt flow 130 may be performed during the hemodialysis session when this shunt flow is closely monitored by the dialysis machine. The technician may use the flow information from the dialysis machine in order to determine how much fluid to add or withdraw from the control chamber 220 in order to achieve a desired level of the shunt flow 130. For example, during the beginning of the dialysis procedure, fluid may be withdrawn from the control chamber 220 until the shunt flow 130 has reached the level sufficient for initiating the hemodialysis treatment. At the end of the procedure, the same volume of fluid may be injected back into the control chamber 220 to reduce the blood flow back to a pre-dialysis, dormant level.

In further embodiments, the syringe 150 may be equipped with a pressure transducer (not shown) so as to allow detection of the fluid pressure in the control chamber 220, which is indicative of the pressure of the shunt blood flow 130 when the flow modulation device 200 is at least partially inflated.

The present invention provides an advantageous solution to many important fistula-associated medical problems including:
1) Decrease in dialysis-associated steal syndrome and ischemic neuropathy. When the patient is not having dialysis, the flow modulation device 200 may be partially or completely inflated and the diameter DD of the arteriovenous connection 180 is reduced, thereby most of the blood flow 110 in the feeding artery 100 favors traveling into the distal artery and past the arteriovenous connection 180 illustrated. This maximizes the blood flow 140 in the distal artery, improves perfusion of the hand, and ameliorates the symptoms of the steal and neuropathy.
2) Decrease the incidence of high-output heart failure. When the patient is not having dialysis, the flow modulation device 200 is inflated and the blood flow 130 in the fistula 180 is reduced as illustrated in FIG. 2 and FIG. 4. This means that the blood return to the heart is reduced and there is no demand for an increased cardiac output. The heart is not required to overwork anymore and the incidence of heart failure secondary to high levels of shunt flow 130 is decreased.
3) Increase the longevity of the dialysis fistula or graft. When the patient is not having dialysis and the flow modulation device 200 is inflated to decrease the shunt blood flow 130, the total venous blood flow is reduced causing a reduction in the venous blood pressure. This reduces the risk of high shear stress causing the development of intimal hyperplasia and premature failure of the arteriovenous connection 180.

Unlike other previous devices (U.S. Pat. Nos. 6,585,762, 3,826,257, 9,907,900) which only dealt with a graft connection, the present invention provides a flow modulation solution to any form of an arteriovenous connection (fistula or graft). The other drawback of the previous devices is that the mechanism of action is built into the graft tubing itself. Any malfunction in that mechanism at any point (like the valve or balloon problems) necessitates a total replacement of the entire graft. As opposed to these devices, the present invention uses a separate device, which is easier to replace via only a minimally invasive procedure. An additional advantage of the present invention is that it can be positioned around any vessel in anticipation to control or stop the bleeding in the postoperative period after a critical vascular procedure.

It is contemplated that any embodiment discussed in this specification can be implemented with respect to any method of the invention, and vice versa. It will be also understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20 or 25%.

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A flow modulation device for use with a hemodialysis fistula or a graft, the flow modulation device comprising a non-expandable outer jacket with an inwardly-inflatable flow-modulation chamber positioned inside thereof, the flow modulation chamber is in fluid communication via a flexible catheter with a control chamber comprising a puncture-resistant housing having an inner cavity covered by a self-sealing elastic membrane sealingly attached thereto and configured for repetitive needle punctures, wherein fluid introduced into the control chamber via a needle puncture of the elastic membrane thereof causing inward inflation of the flow modulation chamber and compression of the hemodialysis fistula or the graft when the flow modulation device is positioned around thereof, causing reduction of blood flow therethrough.

2. The flow modulation device as in claim 1, wherein the non-expandable outer jacket is made in a shape of a hollow cylinder.

3. The flow modulation device as in claim 1, wherein the non-expandable outer jacket is made with a C-shaped cross-section.

4. The flow modulation device as in claim 1, wherein the non-expandable outer jacket and the inwardly-inflatable flow-modulation chamber are made flexible and configured to wrap about the hemodialysis fistula or the graft, the non-expandable outer jacket further comprising two strips positioned on opposing sides of the floe modulation chamber and configured for attachment to each other during insertion of the flow modulation device.

5. The flow modulation device as in claim 4, wherein the two strips are configured for suturing, stapling, or snapping to each other.

6. The flow modulation device as in claim 1, wherein the catheter connecting the inwardly-inflatable flow modulation chamber and the control chamber has a reinforced wall configured to resist collapse during fluid withdrawal from the control chamber.

7. A method of modulating shunt blood flow through an arteriovenous connection facilitating hemodialysis, the method comprising the following steps:
a. subcutaneously positioning a flow modulation device comprising a non-expandable outer jacket with an inwardly-inflatable flow modulation chamber positioned inside thereof around the arteriovenous connection,
b. subcutaneously positioning a control chamber fluidly connected to the flow modulation chamber via a flexible catheter, the control chamber comprising puncture-resistant housing having an inner cavity covered by a self-sealing elastic membrane sealingly attached thereto and configured for repetitive needle punctures,
c. gaining access to the control chamber via a needle puncture, and
d. injecting or withdrawing fluid into or out of the control chamber, thereby inflating or deflating the flow modulation chamber around the arteriovenous connection, whereby inflation of the flow modulation chamber causing the flow modulation device to compress the arteriovenous connection and reduce blood flow therethrough.

8. The method as in claim 7, wherein steps (c) and (d) are performed during a hemodialysis treatment procedure using a hemodialysis machine operated to measure the shunt blood flow through the arteriovenous connection, and step (d) further comprising injecting or withdrawing fluid until the desired shunt blood flow level is reached as indicated by the measurement thereof by the hemodialysis machine.

9. The method as in claim 7, wherein the arteriovenous connection is an arteriovenous fistula.

10. The method as in claim 7, wherein the arteriovenous connection is a graft connecting an artery to a vein.

11. The method as in claim 7, wherein step (a) further comprises a step of wrapping the flow modulation device around the arteriovenous connection.

12. The method as in claim 7, wherein the shunt blood flow is increased during hemodialysis treatments and reduced in between these treatments.
